SEQUENCE LISTING (4 sequences)

SEQ ID NO: 1 - DNA, 651 bp, ACINETOBACTER BAUMANNII
  CDS (1)..(651)
atgcgtgcattagttatttcaacagtggtaggggcagcagtagtactttctggttgtcaaacaacaggtaataaccttggtggcgttgaatacgataaagccgcattaggtactttgatcggcgcagcagctggctacggtatttctaaatcaaatgcaaactctagccgtcaaaacaaccgtgctgcggcaattggtgcagttcttggtgcagctggcggtttatatcttgaccaaaaagagaaaaaattacgcgaacaaatggctggtactggtgtagaagtaggccgtaacccagatggttctgttcaattgatcatgcctggtagcattacttttgatactaacaaatcaaacatcaagccaaacttctatgcaactttggacaaagtagctcaaacattggctgaagataacaagagcgcgattttagttactggttatacagataacactggtaatgactctattaacatcccattatctcaagcgcgtgctcaatcagttaaaaactatttagctggtaaaggtgttccatctagccgtatcgatgcacaaggttatggttcttctaacccaatcgcagacaactcaactgcttctggtcgtgaacaaaaccgccgtgtagaaatcagcatttacgctaaacaa

SEQ ID NO: 2 - protein, 217 aa, ACINETOBACTER BAUMANNII
MRALVISTVVGAAVVLSGCQTTGNNLGGVEYDKAALGTLIGAAAGYGISKSNANSSRQNNRAAAIGAVLGAAGGLYLDQKEKKLREQMAGTGVEVGRNPDGSVQLIMPGSITFDTNKSNIKPNFYATLDKVAQTLAEDNKSAILVTGYTDNTGNDSINIPLSQARAQSVKNYLAGKGVPSSRIDAQGYGSSNPIADNSTASGREQNRRVEISIYAKQ

SEQ ID NO: 3 - DNA, 33 bp, Artificial Sequence
  OMP22 Forward Primer
ggaattccatatgatgcgtgcattagttatttc

SEQ ID NO: 4 - DNA, 29 bp, Artificial Sequence
  OMP22 Reverse Primer
gagcaagcttttgtttagcgtaaatgctg

(19) United States
(12) Patent Application Publication
(10) Pub. No.: US 2023/0116883 A1
(43) Pub. Date: Apr. 13, 2023

(54) ANTI-ACINETOBACTER BAUMANNII POLYCLONAL ANTIBODY (AB-pAb), AND USES THEREOF

(71) Applicant: The Regents of the University of California, Oakland (US)

(72) Inventors: Lee Woodland Riley, Berkeley, CA (US); Nicole Jackson, Berkeley, CA (US)

(21) Appl. No.: 17/914,274

(22) PCT Filed: Mar. 30, 2021
(86) PCT No.: PCT/US21/24838

§ 371 Date: Sep. 23, 2022

Related U.S. Application Data
(60) Provisional application No. 63/002,251, filed on Mar. 30, 2020.

(51) Int. Cl. C07K 16/12; G01N 33/569

(52) U.S. Cl. CPC C07K 16/1217; G01N 33/56911; C07K 2317/33; C07K 2317/92; G01N 2333/22; G01N 2469/10

(57) ABSTRACT

The disclosure provides for a polyclonal antibody that specifically detects Acinetobacter baumannii, a multi-drug resistant (MDR) bacterial pathogen, and uses thereof, including as diagnostic tests and for immunoassays to be used in therapeutic decision-making or research experiments.

### CROSS REFERENCE TO RELATED APPLICATIONS

This application claims priority under 35 U.S.C. § 119 from Provisional Application Ser. No. 63/002,251 filed Mar. 30, 2020, the disclosures of which are incorporated herein by reference.

### STATEMENT OF GOVERNMENT SUPPORT

This invention was made with government support under Grant Number AI117064 awarded by the National Institutes of Health. The government has certain rights in the invention

### TECHNICAL FIELD

The disclosure provides for a polyclonal antibody and polyclonal antibody population that specifically detects Acinetobacter baumannii, a multi-drug resistant (MDR) bacterial pathogen, and uses thereof, including as a therapeutic, for diagnostic tests and for immunoassays.

### BACKGROUND

Acinetobacter baumanii was recently designated by the World Health Organization (WHO) to be one of three “priority 1, critical pathogens” in need of new diagnostic tests, drugs, and research. It is a Gram-negative bacterial pathogen, which has become a major multidrug-resistant pathogen causing healthcare infections worldwide.

### SUMMARY

The disclosure provides a polyclonal antibody (pAb) and polyclonal antibody population for the specific detection of the multi-drug resistant (MDR) bacterial pathogen, Acinetobacter baumannii (A. baumannii). The polyclonal antibody entitled ‘AB-pAb’ herein, was raised against a recombinant (His-tagged) 22 kDa outer membrane protein (OMP22), an antigenic protein which is conserved across the species. The gene encoding OMP22 was amplified from the clinical A. baumannii isolate, AR_0056, which belongs to the international clonal lineage II, a lineage associated with outbreaks worldwide. The AB-pAb is capable of recognizing purified, denatured, OMP22 by Western blot, in addition to the native protein in whole cells of A. Baumannii in vitro. The pAb was optimized for diagnostic use by firstly, removing antibodies within the heterogeneous pAb pool which were cross-reactive to other, clinically relevant Gram-negative bacteria (GNB). This eliminates the issue of cross-reactivity often associated with polyclonal antibodies, which can limit their use as diagnostic tools.

Moreover, testing was performed under conditions which mimic those of the blood and urine, further enhancing the AB-pAb's ability to recognize target bacteria in patient samples. When tested against a panel of clinical isolates by indirect-ELISA, for the recognition of A. baumannii from other clinically relevant GNB, the optimized AB-pAb had a sensitivity of 85.5% (95% confidence interval: 76.11% to 92.3%) and a specificity of 99.5% (95% confidence interval: 99.53% to 99.99%) at a cutoff, signal-to-noise ratio (SNR) of 0.1275.

An antibody-based diagnostic test for A. baumannii is further provided herein, which offers major advantages over the traditional biochemical-based tests or modern nucleic-acid amplification-based tests in terms of simplicity, speed, and cost. In a particular embodiment, the disclosure provides for a polyclonal antibody (pAb) which specifically binds to antigen(s) from Acinetobacter baumannii (A. baumannii), wherein the polyclonal antibody shows limited cross-reactivity to antigens from other gram-negative bacteria. In a further embodiment, the polyclonal antibody specifically binds to Omp22 from A. baumannii. In yet a further embodiment, the Omp22 has the polypeptide sequence of SEQ ID NO:2. In another embodiment, the polyclonal antibody shows limited cross-reactivity to gram-negative bacteria selected from Escherichia coli, Klebsiella pneumoniae, Pseudomonas aeruginosa, Citrobacter freundii, Enterobacter cloacae, Enterobacter aerogenes, Klebsiella oxytoca, Morganella morganii, Proteus mirabilis, and Salmonella enterica. In yet another embodiment, the polyclonal antibody was raised and isolated from rabbits, chickens, goats, guinea pigs, hamsters, horses, mice, rats, or sheep. In a further embodiment, the polyclonal antibody was raised and isolated from rabbits.

In a certain embodiment, the disclosure also provides a detecting assay comprising a polyclonal antibody as disclosed herein. In a further embodiment, the detecting assay is selected from immunoassay, agglutination assay, biochip, microarray and thin layer chromatography assay. In yet a further embodiment, the immunoassay is selected from radioimmunoassays, enzyme-linked immunosorbent assays (ELISA), Western blot, immunofluorescent assays, chemiluminescent assays, lateral flow immunoassay and bioluminescent assays. In another embodiment, the immunoassay is an ELISA assay.

In a particular embodiment, the disclosure further provides a method for diagnosing or detecting the presence of A. baumannii in a sample, comprising: (a) contacting a polyclonal antibody disclosed herein with a sample suspected of having A. baumannii, wherein if the sample comprises A. baumannii then the polyclonal antibody forms a complex with A. baumannii bacterium; (b) adding one or more detecting agents that selectively binds with the polyclonal antibody or antigens from A. baumannii; (c) detecting or measuring the signal produced by the one or more detecting agents, wherein if little to no signal is produced indicates that the samples does not comprise A. baumannii. In another embodiment, the one or more detecting agents are a polyclonal antibody disclosed herein which has been engineered to comprise a label, and wherein the signal is produced from the label. In yet another embodiment, the one or more detecting agents comprise a polyclonal antibody disclosed herein that is bound by a secondary antibody that comprises a label. In a further embodiment, the label is selected from the group consisting of an enzyme, a radio-label and a fluorescence-label. In yet a further embodiment, the label is an enzyme selected from horseradish peroxidase (HRP), alkaline phosphatase (AP), β-galactosidase, acetylcholinesterase and catalase; and wherein the signal is produced from the label is from the enzyme converting a substrate into a detectable product. In another embodiment, the polyclonal antibody of step (a) is bound to a solid substrate. In yet another embodiment, the solid substrate is selected from glass, plastics, nylon or nitrocellulose, resins, and silica or silica-based materials. In a further embodiment, the solid substrate is a multi-well (e.g., 96-well or 384-well) polystyrene plate. In yet a further embodiment, the sample is obtained from a human patient. In a certain embodiment, the sample is a blood or urine sample. In another embodiment, the sample is a water or environmental sample.

In a particular embodiment, the disclosure further provides a method for diagnosing or detecting the presence of A. baumannii in a sample, comprising: adding a sample suspected of comprising A. baumannii to a multiwell (e.g., 96-well) polystyrene plate comprising a polyclonal antibody disclosed herein, wherein the polyclonal antibody is bound to the surface of the plate; incubating the sample with the polyclonal antibody for about 1 hour or more; washing the plate with phosphate buffered saline comprising a surfactant/detergent (e.g., 0.05% Tween-20 (PBST)); blocking the plate for about one hour or more with non-specific protein (e.g., 5% non-fat dry milk) in PBST (blocking buffer); washing the plate one or more times with PBST; incubating the plate for about one hour or more with a rabbit raised polyclonal antibody disclosed herein that is diluted in blocking buffer; washing the plate one or more times with PBST; incubating the plate for about one hour or more with and anti-rabbit labeled IgG antibody (e.g., a goat anti-rabbit-IgG-HRP conjugate) in blocking buffer; washing the plate one or more times with PBST; incubating the plate with developer that reacts with the label on the anti-rabbit IgG (e.g., where the label is HRP a peroxidase substrate is used for about 20 minutes or more); optionally stopping the label enzyme reaction (e.g., by adding 1M HCL to stop the HRP peroxidase reaction); and measuring the plate to detect the label (e.g., for HRP at an OD_{450 nm} using a plate reader).

### DETAILED DESCRIPTION

As used herein and in the appended claims, the singular forms “a,” “an,” and “the” include plural referents unless the context clearly dictates otherwise. Thus, for example, reference to “a pathogen specific antibody” includes a plurality of such antibodies and reference to “the diagnostic platform” includes reference to one or more diagnostic platforms and equivalents thereof known to those skilled in the art, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although many methods and reagents are similar or equivalent to those described herein, the exemplary methods and materials are disclosed herein.

All publications mentioned herein are incorporated herein by reference in full for the purpose of describing and disclosing the methodologies, which might be used in connection with the description herein. Moreover, with respect to any term that is presented in one or more publications that is similar to, or identical with, a term that has been expressly defined in this disclosure, the definition of the term as expressly provided in this disclosure will control in all respects.

It should be understood that this disclosure is not limited to the particular methodology, protocols, and reagents, etc., described herein and as such may vary. The terminology used herein is for the purpose of describing particular embodiments only and is not intended to limit the scope of the present invention, which is defined solely by the claims.

Other than in the operating examples, or where otherwise indicated, all numbers expressing quantities of ingredients, temperatures, time periods or reaction conditions used herein should be understood as modified in all instances by the term “about.” The term “about” when used to described the invention, in connection with percentages means ±1% to 5%.

The term “Acinetobacter baumannii” refers to a Gram-negative bacillus bacterium that is aerobic, pleomorphic and non-motile. An opportunistic pathogen, A. baumannii has a high incidence among immunocompromised individuals, particularly those who have experienced a prolonged (>90 d) hospital stay.

The term “antibody” describes a functional component of serum and is often referred to either as a collection of molecules (antibodies or immunoglobulin) or as one molecule (the antibody molecule or immunoglobulin molecule). An antibody molecule is capable of binding to or reacting with a specific antigenic determinant (the antigen or the antigenic epitope), which in turn may lead to induction of immunological effector mechanisms. An individual antibody molecule is usually regarded as monospecific, and a composition of antibody molecules may be monoclonal (i.e., consisting of identical antibody molecules) or polyclonal (i.e., consisting of different antibody molecules reacting with the same or different epitopes on the same antigen or on distinct, different antigens). Each antibody molecule has a unique structure that enables it to bind specifically to its corresponding antigen, and all-natural antibody molecules have the same overall basic structure of two identical light chains and two identical heavy chains. Antibodies are also known collectively as immunoglobulin. The terms antibody or antibodies as used herein is used in the broadest sense and covers intact antibodies, chimeric, humanized, fully human and single chain antibodies, as well as binding fragments of antibodies, such as Fab, Fv fragments or scFv fragments, as well as multimeric forms such as dimeric IgA molecules or pentavalent IgM.

The term “anti-Acinetobacter baumannii recombinant polyclonal antibody” or “AB-pAb” describes a composition of recombinantly produced diverse antibody molecules, where the individual members are capable of binding to at least one epitope of an Acinetobacter baumannii bacterium.

The term “purified anti-Acinetobacter baumannii recombinant polyclonal antibody” or “purified AB-PAB” refers to purified AB-PAB that is essentially free from immunoglobulin molecules that do not bind to Acinetobacter baumannii antigens” means that more than 80% of the antibodies, preferably more than 90%, more preferably more than 95% and most preferably more than 99%, bind to one of the Acinetobacter baumannii antigens.

The terms “a distinct member of an anti-Acinetobacter baumannii recombinant polyclonal antibody” denotes an individual antibody molecule of the recombinant polyclonal antibody composition, comprising one or more stretches within the variable regions, which are characterized by differences in the amino acid sequence compared to the other individual members of the polyclonal protein. These stretches are located in the CDR1, CDR2 and CDR 3 regions.

“Binding affinity” generally refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{d}). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of this disclosure.

A “biological sample” encompasses a variety of sample types obtained from an individual and can be used in a diagnostic or monitoring assay. The definition encompasses blood and other liquid samples of biological origin, solid tissue samples such as a biopsy specimen or tissue cultures or cells derived therefrom, and the progeny thereof. The definition also includes samples that have been manipulated after their procurement, such as by treatment with reagents, solubilization, or enrichment for certain components, such as proteins or polynucleotides, or embedding in a semi-solid or solid matrix for sectioning purposes. The term “biological sample” encompasses a clinical sample, and also includes cells in culture, cell supernatants, cell lysates, serum, plasma, biological fluid, and tissue samples. The source of the biological sample may be solid tissue as from a fresh, frozen and/or preserved organ or tissue sample or biopsy or aspirate; blood or any blood constituents; bodily fluids such as cerebral spinal fluid, amniotic fluid, peritoneal fluid, \ interstitial fluid, or bronchial alveolar lavage fluid; cells from any time in gestation or development of the subject. The biological sample may contain compounds which are not naturally intermixed with the tissue in nature such as preservatives, anticoagulants, buffers, fixatives, nutrients, antibiotics, or the like.

The term “epitope” is commonly used to describe a site on a larger molecule (e.g., antigen) to which the antibody will bind. An antigen is a substance that stimulates an immune response, e.g. bacteria, bacterial proteins or bacterial DNA. An antigen often has more than one epitope, unless they are very small. Antibodies binding to different epitopes on the same antigen can have varying effects on the activity of the antigen they bind depending on the location of the epitope. An antibody binding to an epitope in an active site of the antigen may block the function of the antigen completely, whereas another antibody binding at a different epitope may have no or little effect on the activity of the antigen. Such antibodies, may however still activate complement and thereby result in the elimination of the antigen.

The term “immunoglobulin” commonly is used as a collective designation of the mixture of antibodies found in blood or serum, but may also be used to designate a mixture of antibodies derived from other sources.

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In some embodiments, the antibody will be purified (1) to greater than 95% by weight of antibody as determined by the Lowry method, and typically more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or silver stain. An isolated antibody includes the antibody in situ within recombinant cells since at least one component of the antibody's natural environment will not be present. Ordinarily, however, an isolated antibody will be prepared by at least one purification step.

The term “fully human” used for example in relation to protein sequences describes sequences which are between 98 to 100% human.

The word “label” when used herein refers to a compound or composition which is conjugated or fused directly or indirectly to a reagent such as an antibody and facilitates detection of the reagent to which it is conjugated or fused. The label may itself be detectable (e.g., radioisotope labels or fluorescent labels) or, in the case of an enzymatic label, may catalyze chemical alteration of a substrate compound or composition which is detectable.

The term “polyclonal antibody” describes a composition of different (diverse) antibody molecules which is capable of binding to or reacting with several different specific antigenic determinants on the same or on different antigens. Usually, the variability of a polyclonal antibody is located in the so-called variable regions of the polyclonal antibody, in particular in the CDR regions. In the disclosure a polyclonal antibody may either be produced in one pot from a polyclonal cell line, or it may be a mixture of different polyclonal antibodies. A mixture of monoclonal antibodies is not as such considered a polyclonal antibody, since they are produced in individual batches and not necessarily form the same cell line which will result in e.g. post translational modification differences. However, if a mixture of monoclonal antibodies provides the same antigen/epitope coverage as a polyclonal antibody of the disclosure it will be considered as an equivalent of the polyclonal antibody. When stating that a member of a polyclonal antibody binds to an antigen, it is herein meant a binding having binding constant that is below 100 nM, preferably below 10 nM, even more preferred below 1 nM.

The term “recombinant antibody” is used to describe an antibody molecule or several molecules that is/are expressed from a cell or cell line transfected with an expression vector comprising the coding sequence of the antibody which is not naturally associated with the cell. If the antibody molecules in a recombinant antibody composition are diverse or different, the term “recombinant polyclonal antibody” or “PAB” applies in accordance with the definition of a polyclonal antibody.

The term “recombinant polyclonal cell line” or “polyclonal cell line” refers to a mixture/population of protein expressing cells that are transfected with a repertoire of variant nucleic acid sequences (e.g., a repertoire of antibody encoding nucleic acid sequences). Typically, the transfection is performed such that the individual cells, which together constitute the recombinant polyclonal cell line, each carry a transcriptionally active copy of a single distinct nucleic acid sequence of interest, which encodes one member of the recombinant polyclonal antibody of interest. Even more typical, only a single copy of the distinct nucleic acid sequence is integrated at a specific site in the genome. The cells constituting the recombinant polyclonal cell line are selected for their ability to retain the integrated copy of the distinct nucleic acid sequence of interest, for example by antibiotic selection. Cells which can constitute such a polyclonal cell line can be for example bacteria, fungi, eukaryotic cells, such as yeast, insect cells, plant cells or mammalian cells, especially immortal mammalian cell lines such as CHO cells, COS cells, BHK cells, myeloma cells (e.g., Sp2/0 cells, NS0), NIH 3T3, YB2/0 and immortalized human cells, such as HeLa cells, HEK 293 cells, or PER.C6.

Acinetobacter baumanii was recently designated by the World Health Organization (WHO) to be one of three “priority 1, critical pathogens” in need of new diagnostic tests, drugs, and research. It is a Gram-negative bacterial pathogen, which has become a major multidrug-resistant pathogen causing healthcare infections worldwide.

Pathogen specific polyclonal antibodies (pAbs), such the AB-pAb disclosed herein, have commerciality in both research and diagnostic applications. Antibodies are integral components of a wide variety of techniques used in both research and diagnostics, including immunoassays (enzyme, radio or fluorescence-linked), immunohistochemistry and flow cytometry.

Provided herein is the development of a diagnostic that can detect A. baumanii in a sample. In particular, the diagnostic of the disclosure is an antibody-based diagnostic test, which offers major advantages over the traditional biochemical-based tests or modern nucleic-acid amplification-based tests in terms of simplicity, speed, and cost. An important commercial application of the AB-pAb of the disclosure would be for diagnostic purposes, with particular utility in rapid, point of care (POC) testing devices. Antibodies can be integrated into low-cost diagnostic platforms, (such as microfluidic or paper-based devices, such as lateral flow assays) which enable the rapid identification of bacterial pathogens with a high degree of accuracy, sensitivity and specificity. The use of this type of test for healthcare infections such as those cause by A. baumanii can greatly reduce health care costs. It can also be used by commercial clinical microbiology laboratories for rapid diagnosis as well as by public health reference laboratories for infectious disease surveillance, again with greatly reduced costs.

Polyclonal antibodies comprise a large number of antibodies with different specificities and epitope affinities. For production purposes these antibodies are generally purified from the serum of immunized animals where the antigen of interest stimulates the B-lymphocytes to produce a diverse range of immunoglobulin's specific to that antigen.

Monoclonal antibodies represent a single B lymphocyte generating antibodies to one specific epitope.

Polyclonal antibodies are poly-specific in that a polyclonal antibody preparation is composed of many different antibodies, each recognizing a distinct epitope of one antigen. While in some uses, such as simple detection of a specific antigen, the fact that a polyclonal antibody preparation will bind to more than one epitope on a specific antigen is not an issue.

Monoclonal antibodies are produced from an immortal cell line and therefore it is possible to produce unlimited quantities of highly specific antibodies. Furthermore, all batches will be identical and specific to just one epitope, which is generally considered to be a particular advantage for therapeutic treatments.

Polyclonal antibodies have a significant advantage in that they are less sensitive to antigen changes than monoclonal antibodies. As such, polyclonal antibodies are more tolerant to mutations and/or effective against different strains and/or isolates, by virtue of recognizing multiple epitopes.

For example, while monoclonal antibodies against the cell wall binding domains of the C. difficile toxin proteins have demonstrated neutralizing capabilities, their activity in cell-based assays is significantly weaker than that observed for polyclonal antibody mixtures (Corthier, G. et al., Infect. Immun. 59:1192-1195 (1991); D. F. M. Thomas et al., The Lancet, pp 78-79 (Jan. 9, 1982); Lyerly, D. M. et al., J. Clin. Micro. 21: 12-14 (1985)).

Methods of producing monoclonal or polyclonal antibodies that react specifically with antigens can be prepared using standard methodologies. For example, preparation of monoclonal antibodies by immunizing mice with an appropriate immunogen is described in, e.g., Coligan, Current Protocols in Immunology (1991): Harlow & Lane, ANTIBODIES, A LABORATORY MANUAL, Cold Spring harbor Publication, New York (1988); Goding, Monoclonal Antibodies: Principles and Practice (2d ed. 1986); Kohler & Milstein, Nature 256:495497 (1975). Antibody preparation by selection of antibodies from libraries of nucleic acids encoding recombinant antibodies packaged in phage or similar vectors is described in, e.g., Huse, et al., Science 246:1275-1281 (1989) and Ward, et al., Nature 341:544-546 (1989). In addition, antibodies can be produced recombinantly using methods known in the art and described in e.g., Sambrook, et al., Molecular Cloning, A laboratory Manual (2nd ed. 1989); Kriegler, Gene Transfer and Expression: A Laboratory Manual (1990); and Current Protocols in Molecular Biology (Ausubel et al., eds., 1994).

Antibodies of the disclosure can be further modified by generation of antibody fragments, humanizing, primatizing, or chimerizing the antibody. Once an antibody of appropriate specificity and affinity has been obtained for use as the first antigen binding moiety (e.g., Omp22), the antibody can be conjugated to the second antigen binding moiety.

Antibody fragments suitable for use with the disclosure include any antibody fragment capable of specifically binding to a particular antigen (e.g., Omp22) on the surface of an A. baumanii bacterium. Non-limiting exemplary antibody fragments may include: F(ab′)₂, Fab, Fv, single chain Fv (scFv), dsFv, V_{L} and V_{H} (see, e.g., Fundamental Immunology (Paul ed., 4d ed. 1999); Bird, et al., Science 242:423 (1988); and Huston et al., Proc. Natl. Acad. Sci. USA 85:5879 (1988)). The antibody fragments can be obtained by a variety of methods, including, digestion of an intact antibody with an enzyme, such as pepsin (to generate (Fab′)₂ fragments) or papain (to generate Fab fragments); or de novo synthesis. Antibody fragments can also be synthesized using recombinant DNA methodology. In some embodiments F(ab′)₂ fragments that specifically bind an antigen expressed on the surface of an A. baumanii bacterium.

As mentioned above, humanized antibodies may be generated from the antibodies disclosed herein. Humanized antibodies are antibodies in which the antigen binding loops, i.e., CDRs, obtained from the V_{H} and V_{L} regions of a non-human antibody are grafted to a human framework sequence. Humanization, i.e., substitution of non-human CDR sequences for the corresponding sequences of a human antibody, can be performed following the methods described in, e.g., U.S. Pat. Nos. 5,545,806; 5,569,825; 5,633,425; 5,661,016; Riechmann et al., Nature 332:323-327 (1988); Marks et al., Bio/Technology 10:779-783 (1992); Morrison, Nature 368:812-13 (1994); Fishwild et al., Nature Biotechnology 14:845-51 (1996). Transgenic mice, or other organisms such as other mammals, may also be used to express humanized or human antibodies, as disclosed in U.S. Pat. No. 6,673,986

The antibodies of the disclosure are useful for detecting the presence of A. baumanii in a sample suspected of containing such bacteria. The antibodies disclosed herein are contacted with the sample for a period of time and under assay conditions sufficient for the antibodies to bind to A. baumanii bacteria present in the sample. Such time and conditions can be readily determined by persons skilled in the art, given the teachings described herein. One then determines if the antibodies have bound to A. baumanii bacteria, forming antibody/A. baumanii bacteria complexes. The binding of A. baumanii bacteria to the antibodies is determined by techniques known to those skilled in the art in conjunction with the teachings disclosed herein. This methodology can also be adapted by standard techniques to measuring the amount or concentration of A. baumanii bacteria in the sample.

The antibodies disclosed herein may be coupled to an insoluble or soluble substrate. Soluble substrates include proteins, such as bovine serum albumin. In a particular embodiment, the antibodies of the disclosure are bound to an insoluble substrate, i.e., a solid support. The antibodies should be bound to the support in an amount and manner that allows sufficient binding to A. baumanii bacteria for detection. The actual concentration of the antibodies to a given substrate will depend upon the particular substrate, and the binding efficiency of the antibody to A. baumanii bacteria as well as the intended used, sample, or assay type. The antibodies may be bound to the substrate in any suitable manner. Covalent, noncovalent, or ionic binding may be used. Covalent bonding can be accomplished by attaching the antibodies to reactive groups on the substrate directly or through a chemical linker.

The solid support may be any insoluble material to which the antibodies of the disclosure can be bound and which may be conveniently used in the assay of the disclosure. Such substrates include permeable and semipermeable membranes, glass beads, plastic beads, latex beads, plastic microtiter wells, agarose, dextran, sepharose, and diatomaceous earth. Alternatively, the antibodies may be bound to any porous or liquid permeable material, such as a screen or net. A binder may be used so long as it does not interfere with the ability of the antibodies to bind to A. baumanii bacteria.

After the sample has been contacted with the substrate containing the antibodies for a sufficient period of time to allow A. baumanii bacteria to bind to the antibodies, such binding is detected through the application of an appropriate detecting process or agent. Generally, the substrate is washed to remove all or substantially all unbound materials. The detecting assay may be an immunoassay, agglutination assay, thin layer chromatography assay, or lateral flow assay. Immunoassays include radioimmunoassays, enzyme-linked immunosorbent assays (ELISA), Western blot, immunofluorescent assays, chemiluminescent assays, lateral flow immunoassay and bioluminescent assays. Agglutination assays include liposome agglutination assays and latex agglutination assays. The degree or amount of binding can also be determined through the application of known techniques, providing a measurement of the amount or concentration of A. baumanii bacteria in the sample

In a certain embodiment, the capture and detection antibodies for ELISA assays are obtained by immunizing a suitable host animal, such as a rabbit, with antigens from A. baumanii. In a further embodiment, capture and detection antibodies are obtained from a host animal after injection with Omp22 from A. baumanii. Typically, the capture and/or detector antibodies are obtained from animals demonstrating about a 40%-50% or greater inhibition of the binding of a F(ab)′₂ detector antibody detected in a test binding assay compared to the binding of the detector antibody in a control binding assay. The test binding assay comprises following steps: providing an antigen binding surface comprising anti-A. baumanii rabbit IgG capture antibody, contacting the antigen binding surface with serum obtained from a vaccinated animal (typically, a rabbit) and a positive control comprising Omp22 from A. baumanii in a manner effective to bind the Omp22 antigen to the capture antibody, contacting the bound Omp22 antigen with a detector antibody comprising a biotin moiety and the F(ab)′₂ portion of the anti-A. baumanii host animal antibody separated from (or without) the F₀ portion, and detecting the bound detector antibody. The test binding assay is the same as the control binding assay, except that the serum is obtained from a non-vaccinated rabbit. The percent-inhibition is defined as the amount of bound detector antibody detected in the test binding assay divided by the amount of bound detector antibody detected in the control binding assay (e.g., ODₜₑₛₜ/OD_{control}).

Once isolated from the animal, the anti-A. baumanii antibodies are attached to an immunoassay detection plate by any suitable method. Such attachment can be accomplished by the physical adsorption of the anti-A. baumanii antibody to the surface, by action of van der Waals forces or hydrophobicity or the like. It is generally known among those skilled in the art that proteins generally, and certainly antibodies, have an affinity for plastic or glass surfaces, which are typical surfaces used in the context of the disclosure. Other common surfaces include polymers, both natural, such as cellulose or chitin and the like, and synthetic, such as nylon and the like. The typical surface used in the context of the disclosure is a plastic surface. One could also attach the anti-A. baumanii antibody to the surface by use of reactive groups that are themselves attached to the surface and that react covalently to the anti-A. baumanii antibody.

The anti-A. baumanii antibody of the disclosure is an antibody derived from a first animal, such as a rabbit, wherein the antibody binds to Omp22 from A. baumanii. Optionally, the A. baumanii antibody can be modified by removing the F_{c} or F(ab)′₂ portion, as described in detail with respect to the detector antibody below. The A. baumanii binding surface can have any suitable concentration of the A. baumanii antibody.

Non-specific binding to an antigen binding surface is blocked by contacting the detector plate with a blocking composition. Typically, the blocking composition is substantially free of bovine serum albumin (BSA). The antigen binding surface comprising an anti-A. baumanii antibody is typically contacted with a blocking agent prior to contact with an analyte sample for antigen-binding analysis. The blocking agent is desirably provided as an excess of a suitable compound that will attach to the antigen binding surface in a manner that substantially reduces or prevents non-specific antigen binding (i.e., antigen binding to the surface other than to the bound capture antibody). Preferably, the blocking agent does not itself attract specific or nonspecific attraction of the antigen of interest or antibody directed thereto. In a particular embodiment, the blocking composition is a solution comprising plant-derived proteins, such as Starting Block™ (Pierce Biotechnology, Inc.).

After contacting the antigen binding surface with the blocking composition, an analyte is placed in contact with the antigen binding surface. The analyte is typically a physiological specimen containing A. baumanii, a positive control known to contain A. baumanii, or a negative control known to not contain A. baumanii. The physiological specimen used in the context of the disclosure is any specimen that may be collected from a patient or environmental sample. Typically, the analyte is either a fluid when removed from the patient or macerated or soaked in a physiological saline buffer. Examples of physiological specimens include, but are not limited to, serum, urine, cerebrospinal fluid, bronchoalveolar lavage fluid, pleural fluid, pericardial fluid, peritoneal fluid, synovial fluid, ocular fluid, and abscess contents.

Detection antibodies (e.g., labelled antibodies) are combined with an excess of animal serum, such as Normal Rabbit Serum (NRS), prior to contact with the anti-A. baumanii antibody on an antigen binding surface. The animal serum is selected by a screening method based on the use of an anti-animal antibody as a positive control. The animal serum is obtained from a different animal of the same species as the source of the detector and/or A. baumanii antibodies. For example, the serum is derived from a first rabbit, screened by use of a goat anti-rabbit antibody.

Preferably, the binding of the detector antibody to the bound antigen is not reduced by the presence of the serum. The serum is typically derived from a different animal of the same animal species as the source of the detector antibody and/or the capture antibody. The immunoassay can therefore comprise the step of performing a serum screening assay to identify serum samples that desirably reduce false positives, as indicated by the ability of the serum to increase detector antigen binding to anti-A. baumanii antibody.

In another embodiment, the detection antibody is a modified IgG antibody that does not comprise the crystalline F₀ domain. Immunoglobulin structure consists of an antigen-binding domain (“F(ab)′₂”) and a highly conserved crystalline domain (“F_{c}”), which can be separated by proteolytic digestion with papain to obtain the F₀ fragment or pepsin to obtain the F(ab)′₂ fragment. The F₀ fragment has a very similar amino acid sequence among all immunoglobulin G (“IgG”) molecules of at least the same species; in contrast, the F(ab)′₂ portion has both hypervariable as well as highly conserved regions when compared from antibody to antibody. The F(ab)′₂ portion comprises two F(ab) fragments paired due to certain disulfide bonds that serve to form the F(ab)′₂ structure. Accordingly, a detector antibody that can be used in the context of the present disclosure is a F(ab) or F(ab)′₂ fragment.

In general, the anti-A. baumanii antibody, the detector antibody, and the animal serum can be derived from the same or different animals that have an immune system, which animals are individually of the same or different species. In a particular embodiment, the species of the animal in which the capture antibody is raised is typically derived from a polyclonal preparation from the host animal that is immunized.

Optionally, a portion of the detector antibody itself can be detected. Typically, a portion of the detector molecule is capable of high-affinity binding to a reporter molecule that can be readily detected. For example, the detector antibody can be adapted to bind to a reporter molecule by combining the detector antibody with a biotin moiety to form a high affinity link with a reporter element comprising a streptavidin moiety. Other linking means for joining a reporter element to the detector antibody include, without limitation, sulfosuccinimidyl-4-N-maleimidomethyl-cyclohexane-1-carboxylate (Sulfo-SMCC), sulfosuccinimidyl-6-3′-2-pyridyldithio-propionamido-hexanoate (Sulfo-LC-SPDP), N-maleimidobutyrloxy-sulfo-succinimide ester (Sulfo-GMBS), and the like; two complementary segments of DNA; and a lectin and an appropriate sugar. Unbound detection antibodies can be removed by washing the surface with a wash solution, commonly a neutral saline solution. The wash step at this point in the ELISA protocol is optional depending on whether the protocol is a heterogeneous or homogeneous format.

After contacting a suitable detection enzyme composition with an antigen binding surface under conditions permitting the detection antibody to bind to the surface bound antigen, a composition comprising a reporter element molecule can be contacted with the bound detection antibody. The reporter element molecule is preferably adapted to bind to the detector antibody with a high affinity. For example, when a biotinylated detector antibody is used, a streptavidin-bound reporter element molecule such as horseradish peroxidase can be used. The reporter element composition is added under conditions to permit, or preferably to promote, binding of the detector antibody to the reporter element molecule to form a reporter-conjugated matched pair molecule. Subsequently, unbound reporter-conjugated matched pair molecule can be removed by washing the surface with the same or similar wash solution. This wash step is commonly performed for either hetero- or homogeneous ELISA formats, as the enzyme conjugated to the matched pair (e.g., the biotin-streptavidin combination) is the signal generator by which the ELISA test is assessed.

The reporter element-conjugated matched pair component can include an enzyme or a label that generates a signal by itself (in the case of a fluorescent or radioactive tag) or in the presence of a substrate (in the case of certain enzymes), which signal is commonly a pigment, or visible light, or fluorescence, or radioactivity. Examples of such enzymes include, but are not limited to, a peroxidase, alkaline phosphatase, beta-galactosidase, chloramphenicol acetyl transferase, and/or a luciferase (e.g., that of renilla or a firefly). Examples of substrates for such enzymes include, but are not limited to, luciferin, tetramethylbenzidine, diethanolamine, p-nitrophenol phosphate (PNPP), 2,2′-azino-bis [ethylbenzthiazoline-6-sulfonic acid] (ABTS), o-phenylenediamine dihydrochloride (OPD), 2-Nitrophenyl-b-D-galactopyranoside (ONPG), 4-Nitrophenyl-b-D-glucuronide (NPG). Dyes, fluorescent tags, metal tags, radioactive tags, and the like, include: fluorescein, rhodamine, Texas Red, Cy dyes, R-phycoerythrin, gold, PBXL, magnetic microparticle, and latex microparticle, each of which can be covalently linked to a component of either component of the matched pairs. The signal generated by using said enzymes or labels, includes any or all of detecting or measuring light or radioactive emission, dye generation, color change, magnetic or metallic bound components, light scattering, and the like.

Various types of samples can be tested for the presence of A. baumanii bacteria in accordance with the disclosure. The sample may be a biological sample, comprising or derived from a bodily fluid (e.g., blood, plasma, saliva, sputum, urine) as well as bronchial alveolar lavage fluid, or tissue extract from a human or animal patient.

The disclosure further provides that the antibodies of the disclosure can be used to test for the presence of A. baumanii bacteria in environments that should be sterile or semi-sterile, such as food manufacturing facilities, hospital environments, and food samples. Samples can be collected and prepared for analysis by various means known to those skilled in the art.

In another embodiment, an article of manufacture is provided. The article may include packaging material containing antibodies of the disclosure. The packaging material may include a label or package insert indicating that the biomolecules can be used for detecting the presence of A. baumanii in a sample.

In yet another embodiment, an array is provided. The array may include a substrate having a plurality of addresses, each address having disposed thereon a set of one or more antibodies that specifically interact with A. baumanii and optionally other pathogens.

The methods of the disclosure can be used with an array (i.e., “biochip” or “microarray”) that includes immobilized antibodies that facilitate the detection of a A. baumanii in a biological sample.

The term “array,” as used herein, generally refers to a predetermined spatial arrangement of binding islands, biomolecules, or spatial arrangements of binding islands or biomolecules. Arrays according to the disclosure that include biomolecules immobilized on a surface may also be referred to as “biomolecule arrays.” Arrays according to the disclosure that comprise surfaces activated, adapted, prepared, or modified to facilitate the binding of biomolecules to the surface may also be referred to as “binding arrays.” The disclosure also contemplates surfaces bearing multiple arrays, referred to as “multiple arrays” or “repeating arrays.” The use of the term “array” herein may encompass biomolecule arrays, binding arrays, multiple arrays, and any combination thereof, the appropriate meaning will be apparent from context. The biological sample can include fluid or solid samples from any tissue of the body including plasma.

An array of the disclosure or a solid phase comprises a substrate. By “substrate” or “solid support” or other grammatical equivalents, herein is meant any material appropriate for the attachment of biomolecules and is amenable to at least one detection method. As will be appreciated by those in the art, the number of possible substrates is very large. Possible substrates include, but are not limited to, glass and modified or functionalized glass, plastics (including acrylics, polystyrene and copolymers of styrene and other materials, polypropylene, polyethylene, polybutylene, polyurethanes, TEFLON®, etc.), polysaccharides, nylon or nitrocellulose, resins, silica or silica-based materials including silicon and modified silicon, carbon, metals, inorganic glasses, plastics, ceramics, and a variety of other polymers. In addition, as is known the art, the substrate may be coated with any number of materials, including polymers, such as dextrans, acrylamides, gelatins or agarose. Such coatings can facilitate the use of the array with a biological sample derived from serum.

A planar array of the disclosure will generally contain addressable locations (e.g., “pads”, “addresses,” or “micro-locations”) of biomolecules in an array format. The size of the array will depend on the composition and end use of the array. Arrays containing from about 2 different biomolecules to many thousands can be made. In some embodiments, the compositions of the disclosure may not be in an array format; that is, for some embodiments, compositions comprising a single biomolecule may be made as well. In addition, in some arrays, multiple substrates may be used, either of different or identical compositions. Thus, for example, large planar arrays may comprise a plurality of smaller substrates. Parallel microfluidic devices comprising arrays would be useful for parallel measurements of A. baumanii content of a biological sample.

As an alternative to planar arrays, bead-based assays in combination with flow cytometry have been developed to perform multiparametric immunoassays. In bead-based assay systems the biomolecules can be immobilized on addressable microspheres. Each biomolecule for each individual immunoassay is coupled to a distinct type of microsphere (e.g., “microbead”) and the immunoassay reaction takes place on the surface of the microspheres. Dyed microspheres with discrete fluorescence intensities are loaded separately with their appropriate biomolecules. The different bead sets carrying different capture probes can be pooled as necessary to generate custom bead arrays. Bead arrays are then incubated with the sample in a single reaction vessel to perform the immunoassay.

Product formation of the biomarker with their immobilized capture biomolecules can be detected with a fluorescence-based reporter system. Biomarkers can either be labeled directly by a fluorogen or detected by a second fluorescently labeled capture biomolecule. The signal intensities derived from captured biomarkers are measured in a flow cytometer. The flow cytometer first identifies each microsphere by its individual color code. Second the number of captured biomarkers on each individual bead is measured by the second color fluorescence specific for the bound target. This allows multiplexed quantitation of multiple targets from a single sample within the same experiment. Sensitivity, reliability and accuracy are compared to standard microtiter ELISA procedures. With bead-based immunoassay systems serum components can be simultaneously quantified from biological samples. An advantage of bead-based systems is the individual coupling of the capture biomolecule to distinct microspheres.

An array of the disclosure encompasses any means for detecting A. baumannii. For example, microarrays can be biochips that provide high-density immobilized arrays of recognition molecules (e.g., antibodies), where binding is monitored indirectly (e.g., via fluorescence).

Arrays and microarrays that can be used with the methods to detect the biomarkers described herein can be made according to the methods described in U.S. Pat. Nos. 6,329,209; 6,365,418; 6,406,921; 6,475,808; and 6,475,809, which are incorporated herein in their entirety. Arrays to detect specific selections of sets of biomarkers described herein can also be made using the methods described herein.

Surfaces useful according to the disclosure may be of any desired shape (form) and size. Non-limiting examples of surfaces include chips, continuous surfaces, curved surfaces, flexible surfaces, films, plates, sheets, tubes, and the like. Surfaces have areas ranging from approximately a square micron to approximately 500 cm². The area, length, and width of surfaces according to the disclosure may be varied according to the requirements of the assay to be performed. Considerations may include, for example, ease of handling, limitations of the material(s) of which the surface is formed, requirements of detection systems, requirements of deposition systems (e.g., arrayers), and the like.

In certain embodiments, it is desirable to employ a physical method for separating groups or arrays of binding islands or immobilized biomolecules: such physical separation facilitates exposure of different groups or arrays to different solutions of interest. Therefore, in certain embodiments, arrays are situated within wells of 96, 384, 1536, or 3456 microwell plates. In such embodiments, the bottoms of the wells may serve as surfaces for the formation of arrays, or arrays may be formed on other surfaces and then placed into wells. In certain embodiments, such as where a surface without wells is used, binding islands may be formed or biomolecules may be immobilized on a surface and a gasket having holes spatially arranged so that they correspond to the islands or biomolecules may be placed on the surface. Such a gasket is preferably liquid tight. A gasket may be placed on a surface at any time during the process of making the array and may be removed if separation of groups or arrays is no longer necessary.

Modifications or binding of antibodies in solution or immobilized on an array may be detected using detection techniques known in the art. Examples of such techniques include immunological techniques such as competitive binding assays and sandwich assays; fluorescence detection using instruments such as confocal scanners, confocal microscopes, or CCD-based systems and techniques such as fluorescence, fluorescence polarization (FP), fluorescence resonant energy transfer (FRET), total internal reflection fluorescence (TIRF), fluorescence correlation spectroscopy (FCS); colorimetric/spectrometric techniques; surface plasmon resonance, by which changes in mass of materials adsorbed at surfaces may be measured; techniques using radioisotopes, including conventional radioisotope binding and scintillation proximity assays (SPA); mass spectroscopy, such as matrix-assisted laser desorption/ionization mass spectroscopy (MALDI) and MALDI-time of flight (TOF) mass spectroscopy; ellipsometry, which is an optical method of measuring thickness of protein films; quartz crystal microbalance (QCM), a very sensitive method for measuring mass of materials adsorbing to surfaces; scanning probe microscopies, such as AFM and SEM; and techniques such as electrochemical, impedance, acoustic, microwave, and IR/Raman detection. See, e.g., Mere L, et al., “Miniaturized FRET assays and microfluidics: key components for ultra-high-throughput screening,” Drug Discovery Today 4(8):363-369 (1999), and references cited therein; Lakowicz J R, Principles of Fluorescence Spectroscopy, 2nd Edition, Plenum Press (1999).

In another embodiment, a pre-packaged diagnostic kit for determining whether a therapy is effective for treating an A. baumanii infection is provided. The kit may include an immunoassay or array as described above, and instructions for using the assays.

Lateral flow assay (LFA) based devices are among very rapidly growing strategies for qualitative and quantitative analysis. Lateral flow assays are performed on a strip, different parts of which are assembled on a plastic backing. These parts include a sample application pad, a conjugate pad, a nitrocellulose membrane and an adsorption pad. The nitrocellulose membrane is further divided into test and control lines. Pre-immobilized reagents at different parts of the strip become active upon flow of liquid sample. Lateral flow assays combine unique advantages of biorecognition probes and chromatography. Lateral flow assays basically combine a number of variants such as formats, biorecognition molecules, labels, detection systems and application. Strips used for lateral flow assays contain four main components: a sample application pad, a conjugate pad, nitrocellulose membranes, and an adsorbent pad.

The sample application pad is typically made of cellulose and/or glass fiber. The sample is applied on this pad to start the assay. Its function is to transport the sample to other components of lateral flow test strip (LFTS). The sample pad should be capable of transportation of the sample in a smooth, continuous and homogenous manner. The sample application pads are sometimes designed to pretreat the sample before its transportation. This pretreatment may include separation of sample components, removal of interfering agents, adjustment of pH, etc.

The conjugate pad is the place where labeled biorecognition molecules are dispensed. The material of conjugate pad should immediately release labeled conjugate upon contact with moving liquid sample. The labeled conjugate should stay stable over entire life span of the lateral flow strip. Any variations in dispensing, drying or release of conjugate can change results of the assay. Poor preparation of labeled conjugate can adversely affect sensitivity of assay. Glass fiber, cellulose, poly-esters and some other materials are used to make conjugate pad for the lateral flow assay. The nature of the conjugate pad material has an effect on release of labeled conjugate and sensitivity of assay.

The membrane (e.g., the Nitrocellulose membrane) effects the sensitivity of the lateral flow assay. Nitrocellulose membranes are available in different grades. Test and control lines are drawn over this piece of membrane. An ideal membrane should provide support and good binding to capture probes (antibodies, aptamers etc.). Nonspecific adsorption over test and control lines may affect results of the assay, thus a good membrane will be characterized by less non-specific adsorption in the regions of test and control lines. Wicking rates of membranes can influence assay sensitivity. These membranes are easy to use, inexpensive, and offer high affinity for proteins and other biomolecules. Proper dispensing of bioreagents, drying and blocking play a role in improving sensitivity of assay.

The adsorbent pad works as a sink at the end of the strip. It also helps in maintaining the flow rate of the liquid over the membrane and stops back flow of the sample. The adsorbent capacity to hold liquid can play a role in results of assay. All these components are fixed or mounted over a backing card.

Various formats can be adopted into the lateral flow assay, including the sandwich format, the competitive format and the multiplex detection format.

In a typical sandwich format, label (enzymes or nanoparticles or fluorescent dyes) coated antibody or aptamer is immobilized at the conjugate pad. This is a temporary adsorption which can be flushed away by flow of any buffer solution. A primary antibody or aptamer against target analyte is immobilized over test line. A secondary antibody or probe against labeled conjugate antibody/aptamer is immobilized at control zone. Sample containing the analyte is applied to the sample application pad and it subsequently migrates to the other parts of strip. At the conjugate pad, target analyte is captured by the immobilized labeled antibody or aptamer conjugate and results in the formation of labeled antibody conjugate/analyte complex. This complex now reaches the nitrocellulose membrane and moves under capillary action. At the test line, labeled antibody conjugate/analyte complex is captured by another antibody which is primary to the analyte. The analyte becomes sandwiched between the labeled and primary antibodies forming a labeled antibody conjugate/analyte/primary antibody complex. Excess labeled antibody conjugate will be captured at a control zone by a secondary antibody. Buffer or excess solution goes to absorption pad. The intensity of color at the test line corresponds to the amount of target analyte and is measured with an optical strip reader or visually inspected. Appearance of color at control line ensures that a strip is functioning properly.

A competitive format suits best for low molecular weight compounds which cannot bind two antibodies simultaneously. Absence of color at test line is an indication for the presence of analyte while appearance of color both at test and control lines indicates a negative result. The competitive format has two layouts. In the first layout, solution containing target analyte is applied onto the sample application pad and prefixed labeled biomolecule (antibody/aptamer) conjugate gets hydrated and starts flowing with the moving liquid. The test line contains pre-immobilized antigen (same analyte to be detected) which binds specifically to label conjugate. Control line contains pre-immobilized secondary antibody which has the ability to bind with labeled antibody conjugate. When liquid sample reaches at the test line, pre-immobilized antigen will bind to the labeled conjugate in case target analyte in sample solution is absent or present in such a low quantity that some sites of labeled antibody conjugate were vacant. Antigen in the sample solution and the one which is immobilized at test line of strip compete to bind with labeled conjugate. In another layout, labeled analyte conjugate is dispensed at conjugate pad while a primary antibody to analyte is dispensed at the test line. After application of analyte solution, a competition takes place between analyte and labeled analyte to bind with primary antibody at test line.

Multiplex detection format is used for detection of more than one target species and assay is performed over the strip containing test lines equal to number of target species to be analyzed. It is highly desirable to analyze multiple analytes simultaneously under same set of conditions. Multiplex detection format is very useful in clinical diagnosis where multiple analytes which are inter-dependent in deciding about the stage of a disease are to be detected. Lateral flow strips for this purpose can be built in various ways, i.e., by increasing length and test lines on conventional strip, making other structures like stars or T-shapes.

The biorecognition molecules used with the lateral flow assays described herein include the polyclonal antibody of the disclosure. The polyclonal antibodies disclosed herein are employed on the test and control lines of lateral flow strip and they bind to A. baumanii through immunochemical interactions. Resulting assay is known as lateral flow immunochromatographic assay (LFIA). The polyclonal antibody of the disclosure which specifically binds to A. baumanii is known as primary antibody but the one which is used to bind a target containing designs, formats and applications of lateral flow assay antibody or another antibody is known as secondary antibody.

The list of materials that can be used as a label in a lateral flow assay is extensive and includes gold nanoparticles, colored latex beads, magnetic particles, carbon nanoparticles, selenium nanoparticles, silver nanoparticles, quantum dots, up converting phosphors, organic fluorophores, textile dyes, enzymes, liposomes and others. Any material that is used as a label should be detectable at very low concentrations and it should retain its properties upon conjugation with biorecognition molecules. This conjugation is also expected not to change the features of the bio-recognition probes. The ease in conjugation with biomolecules and stability over longer period of time are desirable features for a good label. Concentrations of labels down to 10™ are optically detectable. After the completion of assay, some labels generate direct signals (as color from gold colloidal) while others require additional steps to produce analytical signals (as enzymes produce detectable product upon reaction with suit-able substrate). Hence the labels which give direct signal are preferable in LFA because of less time consumption and reduced procedure.

Colloidal gold nanoparticles are the most commonly used labels in LFA. Colloidal gold is inert and gives very perfect spherical particles. These particles have very high affinity toward biomolecules and can be easily functionalized. Optical properties of gold nanoparticles are dependent on size and shape. Size of particles can be tuned by use of suitable chemical additives. Their unique features include environment friendly preparation, high affinity toward proteins and biomolecules, enhanced stability, exceptionally higher values for charge transfer and good optical signaling. Optical properties of gold nanoparticle enhance sensitivity of analysis in LFA. Sensitivity is a function of molar absorption coefficient and accumulation of gold nanoparticles on target molecule. Optical signal of gold nanoparticles in colorimetric LFA can be amplified by deposition of silver, gold nanoparticles and enzymes.

Use of magnetic particles as colored labels in LFA has been reported by number of researchers. Colored magnetic particles produce color at the test line which is measured by an optical strip reader but magnetic signals coming from magnetic particles can also be used as detection signals and recorded by a magnetic assay reader. It has been reported that magnetic signals are stable for longer time compared to optical signals and they enhance sensitivity of LFA by 10 to 1000 folds

Fluorescent molecules are widely used in LFA as labels and the amount of fluorescence is used to quantitate the concentration of analyte in the sample. Detection of proteins is accomplished by using organic fluorophores such as rhodamine as labels in LFA. High photostability and brightness are required for LFAs.

Quantum dots are also used in LFAs. These semiconducting particles are not only water soluble but can also be easily combined with biomolecules because of closeness in dimensions. Owing to their unique optical properties, quantum dots have come up as a substitute to organic fluorescent dyes. Like gold nanoparticles QDs show size dependent optical properties and a broad spectrum of wavelengths can be monitored. Single light source is sufficient to excite quantum dots of all different sizes. QDs have high photostability and absorption coefficients. They can retain their fluorescent properties within the cells and bodies of organisms and less susceptible to metabolic degradation because of their inorganic nature.

Upconverting phosphors (UCP) are also labels which find use in LFAs. UPA labels are characterized by their excitation in infra-red region and emission in high energy visible region. Compared to other fluorescent materials, they have a unique advantage of not showing any auto fluorescence. Because of their excitation in IR regions, they do not photo degrade biomolecules. A major advantage lies in their production from easily available bulk materials. UCP particles were found to show size dependent sensitivity and specificity for detection of antibodies using LFA in sera of patients.

Enzymes are also employed as labels in LFA. But they increase one step in LFA which is application of suitable substrate after complete assay. This substrate will produce color at test and control lines as a result of enzymatic reaction. Horse-radish peroxidase labeled antibody conjugates can be used for detection of primary animal IgGs. In case of enzymes, selection of suitable enzyme substrate combination is one necessary requirement in order to get a colored product for strip reader or electroactive product for electrochemical detection. In other words, sensitivity of detection is dependent on the enzyme/substrate combination. Enhanced LFA sensitivity was observed when enzyme loaded gold nanoparticles were used as a label.

Colloidal carbon is comparatively inexpensive LFA label and its production can be easily scaled up. Because of their black color, carbon NPs can be easily detected with high sensitivity. Colloidal carbon can be functionalized with a large variety of biomolecules for detection of low and high molecular weight analytes. Carbon black nanoparticles showed very low detection limits compared to other labels. The sensitivity of LFA employing colloidal carbon is reported to be comparable with ELISA assay.

In case of gold nanoparticles or other color producing labels, qualitative or semi-quantitative analysis can be done by visual inspection of colors at test and control lines. The major advantage of visual inspection is rapid qualitative answer in “Yes” or “NO”. Such quick replies about presence of an analyte in clinical analysis have very high importance. Such tests can help doctors or other investigators to make an immediate decision, e.g., situations where test results from central labs are necessary immediately. But for quantification, optical strip readers are employed for measurement of the intensity of colors produced at test and control lines of strip. This is achieved by inserting the strips into a strip reader and intensities are recorded simultaneously by imaging software. Optical images of the strips can also be recorded with a camera and then processed by using a suitable software. Such systems use monochromatic light and wavelength of light can be adjusted to get a good contrast among test and control lines and background. Automated systems have advantages over manual imaging and processing in terms of time consumption, interpretation of results and adjustment of variables. In case of fluorescent labels, a fluorescence strip reader is used to record fluorescence intensity of test and control lines. Fluorescence brightness of a test line increases with an analyte's concentration in the sample. Magnetic strip readers and electrochemical detectors are also reported as detection systems in LFTS but they are not as common. Selection of detector is mainly determined by the label employed in analysis.

LFA strips give qualitative or semi-quantitative results which can be observed by naked eyes. Conventional LFAs are normally qualitative and give answers as a “yes” or “no” result. A good LFA biosensor should have the following properties: biocompatibility, high specificity, high sensitivity, rapidity of analysis, reproducibility/precision of results, wide working range of analysis, accuracy of analysis, high through-put, compactness, low cost, simplicity of operation, portability, flexibility in configuration, possibility of miniaturization, potential of mass production and on-site detection.

For use in biological applications for detecting an infection by A. baumanii, kits and articles of manufacture are also described herein. Such kits can comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. The containers can be formed from a variety of materials such as glass or plastic.

For example, the container(s) can comprise one or more antibodies described herein. The container(s) optionally have a sterile access port. Such kits comprise an anti-A. baumanii antibody disclosed herein with an identifying description or label or instructions relating to its use in the methods described herein.

A kit will typically comprise one or more additional containers, each with one or more of various materials (such as reagents, optionally in concentrated form, and/or devices) desirable from a commercial and user standpoint for use of a compound described herein. Non-limiting examples of such materials include, but are not limited to, buffers, diluents, filters, needles, syringes; carrier, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded or etched into the container itself; a label can be associated with a container when it is present within a receptacle or carrier that also holds the container, e.g., as a package insert. A label can be used to indicate that the contents are to be used for a specific application. The label can also indicate directions for use of the contents, such as in the methods described herein.

The following examples are intended to illustrate but not limit the disclosure. While they are typical of those that might be used, other procedures known to those skilled in the art may alternatively be used.

### EXAMPLES

Isolate information. The omp22 gene was amplified from the A. baumannii CDC isolate, AR_0056 (GenBank: CP026707.1). The Omp22—full protein sequence can be found under the following locus tag: AM435_18050:

Omp22—full protein (217 AA) Theoretical pI/Mw: 9.30/22485.13

OMP22 amino acid sequence:
(SEQ ID NO: 2)
MRALVISTVVGAAVVLSGCQTTGNNLGGVEYDKAALGTLIGAAAGYGISK
SNANSSRQNNRAAAIGAVLGAAGGLYLDQKEKKLREQMAGTGVEVGRNPD
GSVQLIMPGSITFDTNKSNIKPNFYATLDKVAQTLAEDNKSAILVTGYTD
NTGNDSINIPLSQARAQSVKNYLAGKGVPSSRIDAQGYGSSNPIADNSTA
SGREQNRRVEISIYAKQ
OMP22 nucleotide sequence:
(SEQ ID NO: 1)
ATGCGTGCATTAGTTATTTCAACAGTGGTAGGGGCAGCAGTAGTACTTTC
TGGTTGTCAAACAACAGGTAATAACCTTGGTGGCGTTGAATACGATAAAG
CCGCATTAGGTACTTTGATCGGCGCAGCAGCTGGCTACGGTATTTCTAAA
TCAAATGCAAACTCTAGCCGTCAAAACAACCGTGCTGCGGCAATTGGTGC
AGTTCTTGGTGCAGCTGGCGGTTTATATCTTGACCAAAAAGAGAAAAAAT
TACGCGAACAAATGGCTGGTACTGGTGTAGAAGTAGGCCGTAACCCAGAT
GGTTCTGTTCAATTGATCATGCCTGGTAGCATTACTTTTGATACTAACAA
ATCAAACATCAAGCCAAACTTCTATGCAACTTTGGACAAAGTAGCTCAAA
CATTGGCTGAAGATAACAAGAGCGCGATTTTAGTTACTGGTTATACAGAT
AACACTGGTAATGACTCTATTAACATCCCATTATCTCAAGCGCGTGCTCA
ATCAGTTAAAAACTATTTAGCTGGTAAAGGTGTTCCATCTAGCCGTATCG
ATGCACAAGGTTATGGTTCTTCTAACCCAATCGCAGACAACTCAACTGCT
TCTGGTCGTGAACAAAACCGCCGTGTAGAAATCAGCATTTACGCTAAACA
A

Cloning and transformation of the omp22 gene. PCR protocols were in accordance with the methodology described by (Sambrook and Russell, 2001). DNA was amplified using Q5 polymerase and Q5 reaction buffer (New England Biolabs), and PCR reactions were carried out in volumes of 50 μL. The template genomic DNA of A. baumannii AR_0056 was isolated using the PurElute Bacterial Genomic Kit (Edge BioSystems). The omp22 gene was amplified from the template DNA using the following primers:

### OMP22 Forward NdeI: GGAATTCCATATGATGCGTGCATTAGTTATTTC (SEQ ID NO:3)

### OMP22 Reverse HindIII: GAGCAAGCTTTTGTTTAGCGTAAATGCTG (SEQ ID NO:4)

Annealing temperature: 59° C.

The amplified PCR product and the pET-26b(+) vector (Novagen, EMD Millipore) were adapted for restriction cloning using the enzymes, NdeI and HindIII (New England Biolabs). Sequential digests of the PCR product and vector were performed in accordance with manufacturer's instructions. The digest of the pET-26b(+) vector removed the pelB leader sequence from the multiple cloning site, and allowed the omp22 gene to be cloned in frame with a C-terminal His-tag sequence. The digested PCR product was ligated into the pET-26b(+) vector using T4 DNA ligase (New England Biolabs), via overnight ligation at 4° C., generating the construct pET26b(+):omp22. A ratio of 5:1, vector to insert ratio was used for the ligation. The ligated product was transformed into chemically competent, XL1-Blue competent cells (Invitrogen) before subcloning into OverExpress™ C43 (DE3) (Lucigen) for protein expression, in accordance with manufacturer's instructions. This generated the expression strain, C43(DE3)[pET26b:omp22]. Transformants were selected for on kanamycin (50 μg/mL) containing LB agar plates and grown in kanamycin containing LB broth to maintain the plasmid.

Expression and purification of recombinant, Omp22 for polyclonal antibody development. From a glycerol stock, C43(DE3) [pET26b:omp22] was streaked for single colonies on an LB agar plate (50 μg/mL kanamycin) and grown overnight at 37° C. The following day, a scrape of several distinct colonies was inoculated into pre-warmed LB (37° C.) containing 50 μg/mL kanamycin. The culture was grown at 37° C. with shaking (200 rpm) until an OD600 of 0.6-0.8 was reached. Expression of the recombinant Omp22 protein was induced by addition of 1 mM IPTG. Cultures were induced for 4 hours at 30° C., before cultures were centrifuged (5000×g for 30 minutes at 4° C.). The pellets were resuspended in B-PER Complete protein extraction reagent (Thermo Scientific) to a concentration of 5 mL of reagent/g biomass, containing 1 M NaCl, a protease inhibitor cocktail (complete mini EDTA-free; Roche) and 1 mM PMSF (Thermo Scientific). The pellet resuspension was then frozen overnight (−20° C.). A total of 10 L of induced culture was pelleted and frozen, to ensure enough protein was be produced for antibody development. The protein purification procedure was carried out as follows using the Buffers and Reagents listed in Table 1:

TABLE 1
Buffers and reagents for Omp22 protein purification.

| Buffers and reagents | Notes |
|---|---|
| Lysis buffer | B-PER complete reagent containing |
| | 1M NaCl and 1 mM PMSF |
| Wash buffer | 50 mM NaH2PO4•H2O, 1M NaCl, |
| | 20 mM imidazole |
| Elution buffer | 50 mM NaH2PO4•H2O, 0.5M NaCl, |
| | 250 mM imidazole |
| Ni-NTA resin and polypropylene | From Qiagen |
| gravity flow columns (5 mL) | |
| Sterile, 1 × PBS with 15% | For protein dialysis |
| glycerol, plus dialysis | |
| membrane/cassettes with a 10 KDa | |
| molecular weight cut-off | |

Lysis: The cell suspensions were lysed by freeze-thawing 3 times, before being placed on a rocker for 1 hour at 4° C. Fresh PMSF (1 mM) was added to the culture after the final, freeze-thaw cycle. The cell lysates were then cleared by centrifugation (5000 rpm for 30 minutes) and the pellets discarded.

Purification: The recombinant Omp22 protein was purified from the supernatant under native conditions, using Ni-NTA agarose (Qiagen). The protein purification protocol is provided below. The standard protocol was modified by increasing the NaCl concentration in the wash buffer from 0.3-1 M. No other modifications to the standard, native protein purification protocol was made. The lysate was kept chilled throughout all steps of the purification procedure; all tubes were chilled on ice before lysate/elution was added and the centrifuge was adjusted to 4° C. before use. Afterward, the product was checked for purity by SDS-gel electrophoresis.

Preparation of Ni-NTA resin: 1.5 mL of Ni-NTA agarose was add to a chilled 50 mL tube. A 1.5 mnL slurry of mL Ni-NTA agarose was prepared for each 50 mL of bacterial lysate. 8 mL of chilled lysis buffer was added to the Ni-NTA resin, then centrifuge at 1,000 rpm for 2 minutes. The supernatant was discarded into a waste bath containing bleach or vesphene. this wash step was repeated an additional time. After which, 8 mL of lysis buffer was added to the resin and incubated in a cold room (4° C.) on a rocker for 10 minutes. The tubes containing the resin were centrifuged (1000 rpm for 2 minutes at 4° C.), and then the supernatant discarded. Imidazole was added to the tube, which should be diluted to a concentration of 20 mM upon addition of cell lysate.

Protein purification: Thawed lysate was added to the tube containing Ni-NTA resin. The tube was placed in cold room (4° C.) on a rocker for 1 hour. After centrifuging (1000 rpm for 2 minutes at 4° C.), the supernatant was discarded into a waste bath. The precipitant was washed 5-6 times by adding 12 mL of wash buffer to the resin, centrifuging (1000 rpm for 2 minutes at 4° C.), discarding supernatant each time into a waste bath. During the final wash, the resin and buffer were transferred into a clean, chilled 15 mL falcon tube. The supernatant was discarded leaving the resin in the new tube.

4 mL of elution buffer was then added to the tube. The tube was incubated in the cold room (4° C.) on a rocker for 30 minutes, and then centrifuged (1000 rpm for 2 minutes at 4° C.) to collect the supernatant (Elution 1). The foregoing step was repeated ×2 to generate Elution 2 and 3. Any residual resin from elution fractions was removed by passing over disposable polypropylene columns, by gravity flow. The purification quality of the protein fractions was checked by SDS-PAGE gel electrophoresis. The eluted protein was dialyzed into 1×PBS containing 15% glycerol, at 4° C. where 1 L of buffer was used per 10 mL of eluted protein. The buffer was exchanged twice (1×2-hour incubation, then overnight) to ensure complete removal of elution buffer from sample. The protein sample can be further concentrated, if necessary. Purified Omp22 protein can be stored at 4° C. (short term), or frozen (−20° C.)

Preparation of protein for antibody development. For antibody development, 5 mg of purified protein was prepared at a concentration of 0.4 mg/mL in sterile PBS. Protein preparations had a purity of >90%. Using these preparations, polyclonal antibodies were raised against recombinant Omp22 in lagomorphs (i.e., a rabbits). Two animals were immunized with 0.2 mg of protein. The initial immunization was followed by three booster injections, administered every other week. The resulting polyclonal antibody was purified from serum using a protein A resin. This produced the unoptimized, anti-A. baumannii polyclonal antibody.

Optimization of anti-A. baumannii polyclonal antibody for diagnostic purposes. Antibodies from the polyclonal pool which exhibit cross-reactivities against non-target, Gram-negative bacteria (GNB) were removed from the unoptimized, polyclonal antibody. This was carried out by absorbing the antibody against whole cell, GNB Isolates. Isolates used for antibody absorption were from the ‘SF’ collection, which consists of Gram-negative, BSI isolates obtained from inpatients at the San Francisco General Hospital in California (Adams-Sapper et al., 2013). Isolates were selected for antibody absorption based on the results of preliminary screening with the unoptimized antibody, by indirect-enzyme linked immunoassay (I-ELISA). Iterative rounds of screening were performed against the SF collection, to identify non-target species to which the unoptimized antibody may have off-target activity. Any isolate which gave a positive result was included in the antibody absorption process.

Antibody absorption: Isolates selected for the absorption procedure were inoculated into fresh BHI broth containing bismuth nitrate (0.5 mM) and sodium salicylate (2.5 mM), further labelled herein as ‘capsule inhibitory conditions.’ Under these conditions, planktonic bacteria do not produce capsular polysaccharides, thereby exposing outer membrane proteins for antibody binding. Isolates were then grown overnight (16 hours) at 37° C. with shaking. The following isolates were selected (Species: SF #):

P. aeruginosa: 25, 142, 319, 348, 373, 387, 450, 500, 647, 698 and 827

C. freundii: 46, 49 187 and 242

K. oxytoca: 45, 327, 303, 225, 216 and 524

E. coli: 19 and 200

M. morgannii: 110 and 154

P. mirabilis: 26 and 196

S. enterica: 12 and 489 After overnight growth, 1 mL of each isolate was transferred to an Eppendorf and spun down (13,000 rpm for 1 minute) to obtain the cell pellet. Cell pellets were washed 3 times in sterile PBS. Before the final wash step, pellets of each species were pooled to produce a ‘P. aeruginosa’ pooled cell pellet, ‘C. freundii’ pooled cell pellet, etc. For antibody absorption, the unoptimized antibody was diluted to a concentration of 1 mg/mL in sterile PBS. The antibody was then resuspended with the first of the pooled cell pellets, and incubated at 4° C. for 1 hour, with end-over-end mixing. Following each incubation, the antibody-bacterial suspension was centrifuged (8000×g for 10 minutes). The supernatant (containing un-bound antibody), was then transferred to the next, pooled cell pellet. The pellet containing bound antibodies, was discarded. The process was repeated sequentially against the pooled pellets from each bacterial species. The absorption process was repeated twice against pooled pellets from P. aeruginosa, C. freundii and K. oxytoca species. After final absorption step, the resulting supernatant (containing un-bound antibody, enriched for A. baumannii binding), was filter sterilized (0.2 μM filter) and frozen (−20° C.) in small volumes (˜50 μL) until further use. After the absorption procedure, the resulting optimized antibody was categorized, ‘AB-pAb’.

Validation of AB-pAb—determining antibody specificity and sensitivity by I-ELISA. The AB-pAb was screened by I-ELISA against 24 target (A. baumannii) and 71 non-target, whole cell clinical GNB isolates. Non-target isolates consisted of: Escherichia coli (7 isolates), Klebsiella pneumoniae (8 isolates), Pseudomonas aeruginosa (26 isolates), Citrobacter freundii (5 isolates), Enterobacter cloacae (5 isolates), Enterobacter aerogenes (1 isolate), Klebsiella oxytoca (5 isolates), Morganella morganii (5 isolates), Proteus mirabilis (5 isolates), Salmonella enterica (5 isolates). Each isolate was tested in triplicate.

I-ELISA protocol. Bacterial isolates were sub-cultured in capsule inhibitory conditions and incubated overnight at 37° C. One mL of 10¹⁰ CFU/mL culture was pelleted in a microfuge tube with a microcentrifuge at 13,000 rpm, and the pellet was resuspended in one mL phosphate-buffered saline (PBS, pH 7.4). This cell suspension was used as the inoculum for the indirect ELISA. After inoculation (50 μL), the cell number in the well was 5×10⁸ CFU/mL. After the overnight incubation with live, whole-cell bacteria, the plate was: (1) washed three times with 200 μL of PBS with 0.05% Tween-20 (PBST); (2) blocked for one hour in 250 μL of 5% non-fat dry milk diluted in PBST (blocking buffer); (3) washed; (4) incubated for 1 hour with 50 μL of primary antibody (AB-pAb) diluted in blocking buffer to a final concentration of 4 μg/mL; (5) washed three times with a 5 minute incubation of 200 μL PBST; (6) incubated for 1 hour with 100 μL of goat anti-rabbit-IgG-HRP conjugate (Southern Biotech, Birmingham, Ala.) diluted 1:2500 in blocking buffer; (8) washed with a 5 minute PBST incubation; (9) incubated for 20 minutes in 100 μL with substrate (SureBlue Reserve TMB Microwell Peroxidase Substrate, Kirkegaard and Perry Laboratories, Gaithersburg, Md.); (10) stopped reaction with 100 μL of 1 M HCl; and (11) read on a plate reader (SpectraMax M3, Molecular Devices) at OD_{450 nm}.

Samples were run in triplicate during each I-ELISA test and raw data were used for the analysis. The corrected OD_{450 nm} values were obtained by subtracting primary antibody and secondary antibody-only controls from each corresponding test well. To determine the signal-to-noise ratio (SNR), the corrected OD_{450 nm} value from each test well were divided by the sum of the corrected values from the control wells. The SNRs were used to generate a ROC curve on GraphPad Prism version 7.03 for Windows (GraphPad Software, La Jolla, Calif., www.graphpad.com). The AUC value of the ROC curve was evaluated by the statistical operations in GraphPad Prism version 7.03 at a 95% confidence interval. The sensitivity and specificity of AB-pAb was determined based on the positive cut-off OD₄₅₀ values established from the ROC curve analysis.

It will be understood that various modifications may be made without departing from the spirit and scope of this disclosure. Accordingly, other embodiments are within the scope of the following claims.

1. A polyclonal antibody (pAb) which specifically binds to antigen(s) from Acinetobacter baumannii (A. baumannii), wherein the polyclonal antibody shows limited cross-reactivity to antigens from other gram-negative bacteria.

2. The pAb of claim 1, wherein the polyclonal antibody specifically binds to Omp22 from A. baumannii.

3. The pAb of claim 2, wherein the Omp22 has the polypeptide sequence of SEQ ID NO:2.

4. The pAb of claim 1, wherein the polyclonal antibody shows limited cross-reactivity to gram-negative bacteria selected from Escherichia coli, Klebsiella pneumoniae, Pseudomonas aeruginosa, Citrobacter freundii, Enterobacter cloacae, Enterobacter aerogenes, Klebsiella oxytoca, Morganella morganii, Proteus mirabilis, and Salmonella enterica.

5. (canceled)

6. The pAb of claim 1, wherein the polyclonal antibody was raised and isolated from rabbits.

7. The pAb of claim 4, wherein the pAb shows a mean sensitivity of 85.5% and a mean specificity of 99.5% for A. baumannii in a panel of clinical isolates by indirect-ELISA.

8. A detecting assay comprising the polyclonal antibody of claim 1.

9. The detecting assay of claim 8, wherein the detecting assay is selected from immunoassay, agglutination assay, biochip, microarray and thin layer chromatography assay.

10. The detecting assay of claim 9, wherein the immunoassay is selected from radioimmunoassays, enzyme-linked immunosorbent assays (ELISA), Western blot, immunofluorescent assays, chemiluminescent assays, lateral flow immunoassay and bioluminescent assays.

11. (canceled)

12. A method for diagnosing or detecting the presence of A. baumannii in a sample, comprising:
(a) contacting a polyclonal antibody of claim 1 with a sample suspected of having A. Baumannii, wherein if the sample comprises A. Baumannii then the polyclonal antibody forms a complex with A. Baumannii bacterium;
(b) adding one or more detecting agents that selectively binds with the polyclonal antibody or antigens from A. Baumannii,
(c) detecting or measuring the signal produced by the one or more detecting agents, wherein if little to no signal is produced indicates that the samples does not comprise A. baumannii.

13. The method of claim 12, wherein the one or more detecting agents is a polyclonal antibody to A. baumannii comprising a label, and wherein the signal is produced from the label.

14. The method of claim 12, wherein the one or more detecting agents comprise a secondary antibody that binds to the polyclonal antibody wherein the secondary antibody comprises a label.

15. The method of claim 12, wherein the signal is produced by a label selected from the group consisting of enzyme, radio-label and fluorescence-label.

16. The method of claim 15, wherein the label is an enzyme selected from horseradish peroxidase (HRP), alkaline phosphatase (AP), β-galactosidase, acetylcholinesterase and catalase; and wherein the signal is produced from the label is from the enzyme converting a substrate into a detectable product.

17. The method of claim 12, wherein for step (a), the polyclonal antibody is bound to a solid substrate.

18. The method of claim 12, wherein for the solid substrate is selected from glass, plastics, nylon or nitrocellulose, resins, and silica or silica-based materials.

19. (canceled)

20. The method of claim 12, wherein the sample is obtained from a human patient.

21. The method of claim 20, wherein the sample is a blood or urine sample, or bronchial alveolar lavage fluid.

22. The method of claim 12, where the sample is a water or environmental sample.

23. A method for diagnosing or detecting the presence of A. baumannii in a sample, comprising:
adding a sample suspected of comprising A. baumannii to a 96-well polystyrene plate comprising the polyclonal antibody of claim 1, wherein the polyclonal antibody is bound to the surface of the plate;
incubating the sample with the polyclonal antibody for about 1 hour or more;
washing the plate with phosphate buffered saline comprising 0.05% Tween-20 (PBST);
blocking the plate for about one hour or more with 5% non-fat dry milk in PBST (blocking buffer);
washing the plate one or more times with PBST;
incubating the plate for about one hour or more with a polyclonal antibody of claim 1 diluted in blocking buffer;
washing the plate one or more times with PBST;
incubating the plate for about one hour or more with a secondary antibody labeled with HRP that binds to the IgG domain of the polyclonal antibody;
washing the plate one or more times with PBST;
incubating the plate with peroxidase substrate for about 20 minutes or more;
adding 1M HCL to stop the HRP peroxidase reaction;
measuring the plate at an OD_{450 nm} using a plate reader.
